(19) United States
(12) Patent Application Publication
(10) Pub. No.: US 2012/0039912 A1
(43) Pub. Date: Feb. 16, 2012

(54) RSPONDIN-3 INHIBITION IN BONE DISORDERS

(75) Inventors: Georges Rawadi, Paris (FR); Christof Niehrs, Heidelberg (DE)

(73) Assignees: GALAPAGOS SASU, Romainville (FR); DEUTSCHES KREBSFORSCHUNGSZENTRUM, Heidelberg (DE)

(21) Appl. No.: 13/264,440

(22) PCT Filed: Apr. 13, 2010
(86) PCT No.: PCT/EP10/54817

§ 371 Date: Oct. 14, 2011

Related U.S. Application Data
(60) Provisional application No. 61/169,406, filed on Apr. 15, 2009.

(51) Int. Cl. A61K 39/395; C07H 21/02; A61K 31/7088; A61P 19/08; C12Q 1/68; A61P 35/00; A61P 35/04; C12Q 1/02; G01N 21/78; C12Q 1/66; C07K 16/18; A61P 19/10

(52) U.S. Cl. 424/172.1; 530/389.8; 536/24.5; 514/44 A; 435/29; 436/501; 435/8; 435/7.1; 435/6.11

(57) ABSTRACT

The present invention relates to uses of antagonists of Rspondin-3 (Rspo3) polypeptides or Rspondin-3 nucleic acids. The invention is based on the demonstration that partial deficiency of Rspo3 leads to a significant increase of bone mass. These results indicate a major role for Rspo3 as a bone anabolic marker or target. Thus, the invention also relates to the use of Rspo3 antagonists in the treatment of osteopenia disorders, particularly in conditions associated with increased bone resorption.

### 1. INTRODUCTION

The present invention relates to novel uses of antagonists of Rspondin-3 (Rspo3) polypeptides or Rspo3 nucleic acids. The invention is based on the demonstration that partial deficiency of Rspo3 leads to a significant increase of bone mass. These results indicate a major role for Rspo3 as a bone anabolic marker or target. Thus, the invention also relates to the use of Rspo3 antagonists in the treatment of low bone density disorders, particularly in conditions associated with increased bone resorption and/or decreased bone formation.

### 2. BACKGROUND OF THE INVENTION

The Rspondin protein family is conserved among vertebrates and consists of the four related members Rspondin1-4 (Rspo1-4) (Chen et al., 2002, Mol. Biol. Rep. 29, 287-292, who called Rspo3 hPWTSR; Kamata at al., 2004, Biochim. Biophys. Acta. 1676, 51-62; Kazanskaya et al., 2004, Dev. Cell 7, 525-534; Kim et al., 2005, Science 309, 1256-1259; Kim et al., 2006, Cell Cycle 5, 23-26; Nam et al., 2006, J. Biol. Chem. 281, 13247-13257). Human Rspo1-4 were also described as Stem Cell Growth Factor Like Polypeptides, which are able to promote proliferation of hematopoietic stem cells (WO 01/77169; WO 01/07611). They were also designated as Futrin1-4 and identified as modulators of the Wnt signalling pathway (WO 2005/040418). WO 2007/009105 refers to the use of Rspondin polypeptides, Rspondin nucleic acids or regulators or effectors or modulators for the promotion of angiogenesis and/or vasculogenesis. The content of these documents is herein incorporated by reference and the amino acid and nucleic sequences of Rspondins 1-4 disclosed therein are specifically included herein.

### 3. SUMMARY OF THE INVENTION

The present invention relates to the use of antagonists of Rspo3 polypeptides or Rspo3 nucleic acids, as agents for enhancing bone formation and/or inhibiting bone resorption. According to the present invention it was shown that partial deficiency of Rspo3 in a transgenic animal model results in a significant increase of bone mass. This demonstrates that inhibition of Rspo3 could be a pharmacological approach in bone disorders, in particular in low bone density disorders. Further, a serum marker analysis indicates that Rspo3 is affecting bone formation. Thus secreted Rspo3 might be a bone anabolic target. By administering Rspo3 antagonists low bone density disorders associated with, accompanied by and/or caused by increased bone resorption and/or reduced bone formation may be treated.

In a first aspect, the present invention refers to the use of an antagonist of an Rspondin-3 (Rspo3) polypeptide or an Rspondin-3 (Rspo3) nucleic acid for the manufacture of a medicament for the promotion of bone formation and/or inhibition of bone resorption.

In a further aspect, the present invention refers to a method for promoting bone formation or inhibiting bone resorption comprising administering to a subject in need thereof a therapeutically effective dose of an antagonist of Rspo3 polypeptides or Rspo3 nucleic acids.

In a still further aspect, the present invention refers to a method for the diagnosis or monitoring of bone-associated processes, conditions or disorders, comprising determining the amount, activity and/or expression of an Rspo3 polypeptide or the expression of an Rspo3 nucleic acid in a sample.

In a still further aspect, the invention refers to the use of an Rspo3 polypeptide or an Rspo3 nucleic acid to evaluate and/or screen test compounds for their ability to modulate bone-associated processes, conditions or disorders, wherein an increased activity of said Rspo3 polypeptide or Rspo3 nucleic acid in the presence of the test compound when compared to a control is associated with increased bone resorption and/or decreased bone formation.

In a still further aspect, the invention refers to the use of an Rspo3 polypeptide or an Rspo3 nucleic acid to evaluate and/or screen test compounds for their ability to modulate bone-associated processes, conditions or disorders, wherein a decreased activity of said Rspo3 polypeptide or Rspo3 nucleic acid in the presence of the test compound when compared to a control is associated with decreased bone resorption and/or increased bone formation.

In a still further aspect, the invention refers to the use of a recombinant cell which expresses Rspo3 or non-human transgenic organism exhibiting modified

Rspo3 expression to evaluate and/or screen test compounds for their ability to modulate bone-associated processes, conditions or disorders, wherein an increased amount, activity and/or expression of said Rspo3 polypeptide or Rspo3 nucleic acid is associated with increased bone resorption and/or decreased bone formation.

In a still further aspect, the invention refers to the use of a recombinant cell which expresses Rspo3 or non-human transgenic organism exhibiting modified Rspo3 expression to evaluate and/or screen test compounds for their ability to modulate bone-associated processes, conditions or disorders, wherein a decreased amount, activity and/or expression of said Rspo3 polypeptide or Rspo3 nucleic acid is associated with decreased bone resorption and/or increased bone formation.

### 4. BRIEF DESCRIPTION OF THE FIGURES

FIG. 1: (A) In Rspo3^{+/-} mice the ratio of bone volume to tissue volume (bv/tv) in tibial metaphysis is increased. (B) In Rspo3^{+/-} mice, the trabecular number in tibial metaphysis is increased.

FIG. 2: The amount of the bone formation marker osteocaicin (OCN) is increased in Rspo3^{+/-} mice.

FIG. 3: The Wnt/PCP pathway is activated by Rspo3.

### 5. DESCRIPTION OF THE INVENTION

### 5.1 Definitions

As used herein the term ‘Rspondin3 polypeptide’ or ‘Rspo3 polypeptide’ according to the present invention refers to polypeptides that encode Rspondin3 which may be derived from mammalian or other vertebrate organisms.

Preferably, the Rspondin3 polypeptide is human Rspondin3. The amino acid sequences of human Rspondin3 polypeptide is shown in WO 2005/040418, the content of which is herein incorporated by reference. A particular sequence for human Rspondin3 amino acid sequences is as follows: Human Rspondin-3 amino acid sequence (NP_{—}116173, SEQ ID NO: 1).

Further examples of Rspondin3 sequences are Rspondin3 polypeptide sequences from Xenopus, e.g. Xenopus tropicalis and Xenopus laevis or from Mus musculus.

Rspondin3 polypeptides are further defined herein as polypeptides that show at least 40%, preferably at least 60%, more preferably at least 80%, at least 90%, at least 95%, at least 98% or at least 99% sequence identity at the amino acid level to the respective human Rspondin3 polypeptide over its entire length (Kazanskaya et al., 2004, Dev. Cell 7, 525-534). Further, Rspondin3 polypeptides according to the invention are preferably characterized as having at least one biological activity selected from
i reduction of the ratio bone volume to tissue volume tibial metaphysis and
ii reduction of the trabecular number in tibial metaphysis.
iii inhibition of the non-canonical wnt pathway
The above activities may be determined using any methods known to a person of skill in the art, including but not limited to those methods described herein.

The term ‘polypeptide’ includes full-length proteins, proteinaceous molecules, fragments of proteins, fusion proteins, peptides, oligopeptides, variants, derivatives, analogs or functional equivalents thereof.

The Rspondin3 gene product itself may contain deletions, additions or substitutions of amino acid residues within the Rspo3 sequence, which result in a silent change thus retaining significant signal transducing capacity thus producing a functionally equivalent Rspo3. Such amino acid substitutions may be made on the basis of similarity in polarity, charge, solubility, hydrophobicity, hydrophilicity, and/or the amphipatic nature of the residues involved. For example, negatively charged amino acids include aspartic acid and glutamic acid; positively charged amino acids include lysine and arginine; amino acids with uncharged polar head groups having similar hydrophilicity values include the following: leucine, isoleucine, valine; glycine, analine; asparagine, glutamine; serine, threonine; phenylalanine, tyrosine.

As used herein the term ,, ‘Rspondin3 nucleic acid’ or ‘Rspo3 nucleic acid’ refers to nucleic acid sequences that encode Rspondin3 which may be derived from mammalian or other vertebrate organisms. Preferably, the Rspondin3 nucleic encodes human Rspondin3. The nucleic acid sequences of human Rspondin 1, 2, 3 and 4 are shown in WO 2005/040418, the content of which is herein incorporated by reference. A particular sequence for human Rspondin3 nucleic acid sequences is as follows: Human Rspondin-3 nucleic acid sequence (NM_{—}032784, SEQ ID NO: 2).

Further examples of Rspondin3 nucleic acids are those which encode the Rspondins from Xenopus, e.g, Xenopus tropicalis and Xenopus laevis or from Mus musculus.

Rspondin nucleic acids are further defined herein as molecules selected from
(a) nucleic acid molecules encoding Rspondin3 polypeptides, e.g a human Rspondin3,
(b) nucleic acid molecules which hybridize under stringent conditions to a nucleic acid molecule of (a) and/or a nucleic acid molecule which is complementary thereto,
(c) nucleic acid molecules which encode the same polypeptide as a nucleic acid molecule of (a) and/or (b), and
(d) nucleic acid molecules which encode a polypeptide which is at least 40%, preferably at least 60%, more preferably at least 80%, and most preferably at least 90% identical to a polypeptide encoded by a nucleic acid molecule of (a) over its entire length.

The nucleic acid molecules may be e.g. DNA molecules or RNA molecules.

Nucleic acid molecules which may be used in accordance with the invention may include deletions, additions or substitutions of different nucleotide residues resulting in a sequence that encodes the same or a functionally equivalent gene product.

As used herein, the terms ‘regulators’ or ‘effectors’ or ‘modulators’ of Rspo3 polypeptides or Rspo3 nucleic acids are used interchangeably herein and any of the above may be used to refer to antibodies, peptides, low molecular weight organic or inorganic molecules and other sources of potentially biologically active materials capable of modulating Rspo3 polypeptides, e.g. Rspo3 signal transduction or capable of modulating Rspo3 polypeptide activity or capable of modulating Rspo3 expression to promote (antagonists) or inhibit (agonists) bone formation and/or loss of bone mass. Said regulators, effectors or modulators can be naturally occurring or synthetically produced.

As used herein, the term ‘compound capable of binding to Rspo3’ refers to a naturally occurring or synthetically produced regulator, effector or modulator of

Rspo3 which interacts with an Rspo3 polypeptide. Examples of such compounds are (i) a natural partner, e.g. receptor of an Rspo3; (ii) a naturally occurring molecule which is part of the signalling complex; and/or a naturally occurring signalling molecule produced by other cell types; (iii) naturally occurring or synthetically produced antibody. The term ‘compound’ is used herein in the context of a ‘test compound’ or a ‘drug candidate compound.

As used herein the term ‘agonist of Rspo3’ refers to regulators or effectors or modulators of Rspo3 that activate the intracellular response of Rspo3 and thus promote angiogenesis and/or vasculogenesis.

As used herein, the term ‘antagonist of Rspo3’ refers to regulators or effectors or modulators of Rspo3 polypeptides or Rspo3 nucleic acids that inhibit, decrease or prevent the intracellular response of Rspo3 polypeptides or Rspo3 nucleic acids and thus inhibit, decrease or prevent angiogenesis and/or vasculogenesis.

Examples of suitable antagonists are mutated forms of Rspo3, having a dominant negative effect, Rspo3-binding polypeptides, e.g. anti-Rspo3 antibodies including recombinant antibodies or antibody fragments containing at least one Rspo3 binding site. Further examples of Rspo3 antagonists are nucleic acids capable of inhibiting Rspo3 translation, transcription, expression and/or activity, e.g. aptamers, antisense molecules, ribozymes or nucleic acid molecules capable of RNA interference such as siRNA molecules including nucleic acid analogs such as peptidic nucleic acids or morpholino nucleic acids. Such nucleic acids may bind to or otherwise interfere with Rspondin nucleic acids.

As used herein, the term ‘antibody’ or ‘antibodies’ includes but is not limited to recombinant polyclonal, monoclonal, chimeric, humanized, human, or single chain antibodies or fragments thereof including Fab fragments, single chain fragments, and fragments produced by an Fab expression library. Neutralizing antibodies are especially preferred for diagnostics and therapeutics.

As used herein, the term ‘bone remodelling’ refers to the twin processes of bone formation and bone resorption, in general these processes are balanced, but in some disorders this balance can be lost resulting in a net increase or a net decrease in bone density.

As used herein, the term ‘bone formation’ refers to the process by which osteoblasts deposit a matrix of collagen, whilst also releasing calcium, magnesium, and phosphate ions, which chemically combine and harden within the matrix into the mineral hydroxyapatite.

As used herein, the term ‘bone resorption’ relates to the process by which osteoclasts resorb a discrete area of bone matrix.

As used herein the term ‘modified’ when used with respect to the expression of an Rspo3 polypeptide or an Rspo3 nucleic acid refers to an Rspo3 polypeptide or Rspo3 nucleic acid that is expressed at a different level (e.g. with a higher expression level) that is expressed in a different location (e.g. in a different cell type than where it is usually expressed) or that is expressed at a different time (e.g. in a situation where it is constitutively expressed rather that expressed in response to a particular signal). In particular a cell or non-human transgenic organism that demonstrates modified expression of an Rspo3 nucleic acid or an Rspo3 polypeptide may exhibit permanently modified expression (e.g. due to changes in the genome of the cell or the organism) or it may exhibit transiently modified expression (e.g. due to temporary transfection of an mRNA sequence).

As used herein, the term ‘treating’ or ‘treatment’ refers to an intervention performed with the intention of preventing the development or altering the pathology of, and thereby alleviating a disorder, disease or condition, including one or more symptoms of such disorder or condition. Accordingly, ‘treating’ refers to both therapeutic treatment and prophylactic or preventative measures. Those in need of treating include those already with the disorder as well as those in which the disorder is to be prevented. The related term ‘treatment’, as used herein, refers to the act of treating a disorder, symptom, disease or condition, as the term ‘treating’ is defined above.

As used herein, the term ‘low bone density disorder’ refers to those disorders in which the bone remodelling balance has become distorted resulting in a net decrease in bone density. These disorders may arise as a result of decreased bone formation or increased bone resorption or a combination of both. Particular examples of such disorders include osteoporosis, osteomalacia, nutritional osteopathy, intestinal osteopathy, calcipenic osteopathy, renal osteopathy, osteopenia, bone metastasis (e.g. from lung, breast or prostate origin), osteosarcoma and multiple myeloma. Preferred disorders include osteoporosis, bone metastasis and multiple myeloma.

### 5.2 Detailed Description of the Invention

Bone formation is required for the development and maintenance of mammalian, e.g. human organisms. Decreased bone formation and/or a loss of bone mass (e.g. due to a higher rate of bone resorption) leads to low bone density disorders. The present invention relates to the use of antagonists of Rspo3 polypeptides or Rspo3 nucleic acids for the prevention and/or treatment of low bone density disorders.

The present inventors have found that transgenic mice with a heterozygous Rspo3^{+/-} genotype develop normally and show—compared to control mice—a significant increase of bone mass as evidenced by determining the bone volume/tissue volume ratio and the trabecular number in tibial metaphysis. This effect is found in both female and male organisms.

Further, the heterozygous Rspo3^{+/-} mice exhibited differences in the expression level of bone markers compared to wild-type mice. Particularly, in Rspo3^{+/-} mice, the bone formation marker osteocalcin (OCN) was increased in both female and male organisms.

Accordingly, inhibition of Rspo3 may be useful for the treatment of diseases caused by, associated with and/or accompanied by dysfunctional bone formation and/or increased bone resorption.

An embodiment of the present invention refers to the use of an antagonist of Rspo3 polypeptides or Rspo3 nucleic acids for the manufacture of a bone formation promoting and/or bone loss inhibiting medicament.

Specific disorders which are susceptible to administration of an Rspo3 antagonist include e.g. osteoporosis, osteomalacia, nutritional osteopathy, intestinal osteopathy, calcipenic osteopathy, renal osteopathy and other low bone density disorders such as osteopenia, bone metastasis (e.g. from lung, breast or prostate origin), osteosarcoma, and multiple myeloma.

The antagonists of Rspo3 polypeptides or Rspo3 nucleic acids may be used in human or veterinary medicine, for the treatment of female and/or male subjects, alone or in combination with a further medicament.

In an embodiment of the invention, Rspo3 polypeptides and/or Rspo3 nucleic acids, and/or cell lines or non-human transgenic animals that express an Rspo3 polypeptide or nucleic acid may be used to screen for regulators or effectors or modulators of Rspo3 that act as agonists or antagonists of bone formation. For example, screening to identify antibodies capable of neutralizing the activity of Rspo3, e.g. chimeric antibodies, fully human antibodies, or antibody variable domains, which may be used to inhibit Rspo3 function. Alternatively, screening of peptide libraries or organic compounds with recombinantly expressed soluble Rspo3 polypeptides, cell lines expressing an Rspo3 polypeptide or transgenic non-human animals expressing an Rspo3 polypeptide may be useful for identification of therapeutic molecules that function by modulating, e.g. inhibiting, the biological activity of Rspo3 and thus are suitable as bone formation regulators or effectors or modulators of Rspo3, e.g. antagonists of Rspo3. Alternatively, screening of shRNA libraries or siRNA libraries with cell lines that express an Rspo3 nucleic acid or an Rspo3 polypeptide may be useful for identification of therapeutic molecules that function by modulating, e.g. inhibiting, the expression and/or biological activity of Rspo3 and thus are suitable as bone formation regulators or effectors or modulators of Rspo3, e.g. antagonists of Rspo3.

In an embodiment of the invention, engineered cell lines and/or transgenic non-human animals which exhibit modified Rspo3 expression, e.g. an increased or decreased expression of a Rspo3 polypeptides or Rspo3 nucleic acids compared to wild-type cell lines or animals, may be utilized to screen and identify antagonists as well as agonists. These methods are described in WO 2007/009105, the content of which is herein incorporated by reference.

In a specific embodiment, the present invention relates to a method for identifying a compound that increases bone formation and/or decreases bone resorption, said method comprising:
(a) contacting a cell expressing Rspo3 with a test compound, and
(b) identifying a test compound that increases the expression of marker(s) related to bone formation and/or decreases the expression of marker(s) related to bone resorption.

In one embodiment said method uses mammalian cells.

In one embodiment said method uses cells selected from osteoblasts, and undifferentiated mesenchymal stem cells

In one embodiment the marker(s) related to bone formation are selected from bone alkaline phosphatase, RUNX2, OCN, osteopontin, collagen type I, collagen type II, BMP2 and BMP4.

In a specific embodiment, the present invention relates to a method for identifying a compound that increases bone formation and/or decreases bone resorption, said method comprising:
(a) contacting a cell expressing Rspo3 with a test compound, and
(b) identifying a test compound that decreases the activity of the non-canonical wnt pathway.

In one embodiment the test compound does not inhibit the canonical wnt pathway.

In one embodiment the activity of the non-canonical Wnt pathway (Wnt/PCP pathway) can be measured by Jnk phosphorylation and/or assaying convergent extension movement in Xenopus embryos (Yamanaka et al., 2002, EMBO Rep. 3, 69-75). The activation of the Wnt/PCP pathway can also be measured by ATF luciferase reporter assay in Xenopus embryos.

In one embodiment the activity of the canonical Wnt pathway is measured by TOPFLASH luciferase reporter assays or b-catenin stabilisation arrays (Kazanskaya et al., 2004, Dev. Cell 7, 525-534; Kim et al., Mol. Cell. Biol. 2008, 19, 2588-96).

Various procedures known in the art may be used for the production of antibodies to epitopes of an Rspo3 polypeptide.

Monoclonal antibodies that bind to an Rspo3 polypeptide may be labelled allowing one to follow their location and distribution in the body after injection. Tagged antibodies may be used as a non-invasive diagnostic tool for imaging bone formation and/or resorption associated with conditions where treatment involves inhibiting loss of bone mass and/or promoting bone formation.

Immunotoxins may also be designed which target cytotoxic agents to specific sites in the body. For example, high affinity Rspo3-specific monoclonal antibodies may be covalently complexed to bacterial or plant toxins, such as diptheria toxin, abrin or ricin. A general method of preparation of antibody/hybrid molecules may involve use of thiol-crosslinking reagents such as SPDP, which attack the primary amino groups on the antibody and by disulfide exchange, attach the toxin to the antibody. The hybrid antibodies may be used to specifically eliminate Rspo3 expressing endothelial cells.

For the production of antibodies, various host animals may be immunized by injection with the Rspo3 polypeptide including but not limited to rabbits, mice, rats, etc. Various adjuvants may be used to increase the immunological response, depending on the host species but not limited to Freund's (complete and incomplete), mineral gels such as aluminium hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Monoclonal antibodies to Rspo3 polypeptides may be prepared by using any technique which provides for the production of antibody molecules by continuous cell lines in culture. These include but are not limited to the hybridoma technique originally described by Kohler and Milstein, (Nature, 1975, 256: 495-497), the human B-cell hybridoma technique (Kosbor et al., 1983, Immunology Today, 4: 72; Cote et al., 1983, Proc. Natl. Acad. Sci., 80: 2026-2030) and the EBV-hybridoma technique (Cole et al., 1985, Monoclonal Antibodies and Cancer Therapy, Alan R. Liss, Inc., pp. 77-96). In addition, techniques developed for the production of “chimeric antibodies” (Morrison et al., 1984, Proc. Natl. Acad. Sci., 81: 6851-6855; Neuberger et al., 1984, Nature, 312: 604-608; Takeda at al., 1985, Nature, 314: 452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce Rspo3-specific single chain antibodies.

Antibody fragments which contain specific binding sites for Rspo3 may be generated by known techniques. For example, such fragments include but are not limited to: the F(ab′)₂ fragments which can be produced by pepsin digestion of the antibody molecule and the Fab fragments which can be generated by reducing the disulfide bridges of the F(ab′)2 fragments. Alternatively, Fab expression libraries may be constructed (Huse et al., 1989, Science, 246: 1275-1281) to allow rapid and easy identification of monoclonal Fab fragments with the desired specificity to Rspondin.

Antibodies to Rspo3 polypeptides may antagonise the activity of Rspondin by preventing it from binding to its partners in a signalling cascade. Therefore, antibodies which bind specifically to Rspo3, may be antagonists of Rspo3 which may be used to promote bone formation and/or inhibit bone resorption.

In addition, mutated forms of Rspo3, having a dominant negative effect, may be expressed in targeted cell populations to inhibit the activity of endogenously expressed wild-type Rspo3.

Included in the scope of the invention are nucleic acid antagonists of Rspo3. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of the Rspondin nucleotide sequence, are preferred.

Ribozymes are enzymatic RNA molecules capable of catalyzing the specific cleavage of RNA. The mechanism of ribozyme action involves sequence-specific hybridization of the ribozyme molecule to complementary target RNA, followed by a endonucleolytic cleavage. Within the scope of the invention are engineered hammerhead motif ribozyme molecules that specifically and efficiently catalyze endonucleolytic cleavage of Rspo3 RNA sequences.

Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

RNAi molecules are double-stranded RNA molecules or analogues thereof capable of mediating RNA interference of a target mRNA molecule, e.g. siRNA molecules which are short double-stranded RNA molecules with a length of preferably 19-25 nucleotides and optionally at least one 3′-overhang or precursors thereof or DNA molecules coding therefor. Anti-sense RNA and DNA molecules, ribozymes and RNAi molecules of the invention may be prepared by any method known in the art for the synthesis of RNA molecules. These include techniques for chemically synthesizing oligodeoxyribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various modifications to the DNA molecules may be introduced as a means of increasing intracellular stability and half-life. Possible modifications include but are not limited to the addition of flanking sequences of Morpholino derivatives as well as ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

In a particular embodiment of the invention antagonists of Rspo3 polypeptides or Rspo3 nucleic acids may be used in the treatment of conditions where treatment involves promoting bone formation and/or inhibiting bone resorption, e.g. in osteoporosis, osteomalicia, nutritional osteopathy, intestinal osteopathy, calcipenic osteopathy, renal osteopathy and other low bone density disorders.

In a particular embodiment of the invention the Rspo3 polypeptide antagonist is an Rspo3 antibody. In a most particular embodiment of the invention an Rspo3 antibody may be used to treat conditions wherein treatment involves promoting bone formation and/or inhibiting bone resorption, e.g. osteoporosis, osteomalicia, nutritional osteopathy, intestinal osteopathy, calcipenic osteopathy, renal osteopathy and other low bone density disorders.

In a particular embodiment of the invention the Rspo3 nucleic acid antagonist is a nucleic acid capable of inhibiting Rspo3 translation, transcription, expression and/or activity. In a most particular embodiment of the invention a nucleic acid capable of inhibiting Rspondin translation, transcription, expression and/or activity may be used to treat conditions wherein treatment involves promoting bone formation and/or inhibiting bone resorption, e.g. osteoporosis, osteomalicia, nutritional osteopathy, intestinal osteopathy, calcipenic osteopathy, renal osteopathy and other low bone density disorders. In a most particular embodiment of the invention an siRNA, shRNA or other antisense nucleic acid against Rspo3 may be used to treat conditions where treatment involves promoting bone formation and/or inhibiting bone resorption, e.g. osteoporosis, osteomalicia, nutritional osteopathy, intestinal osteopathy, calcipenic osteopathy, renal osteopathy and other low bone density disorders.

Pharmaceuticay active antagonists of Rspo3 polypeptides or Rspo3 nucleic acids can be administered to a patient either by itself, or in pharmaceutical compositions where it is mixed with suitable carriers or excipient(s).

Depending on the specific conditions being treated, these agents may be formulated and administered systemically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections, or, in the case of solid tumors, directly injected into a solid tumor. For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

The antagonists of Rspo3 polypeptides or Rspo3 nucleic acids can be formulated readily using pharmaceutically acceptable carriers well known in the art into dosages suitable for oral administration. Such carriers enable the active agents of the invention to be formulated as tablets, pills, capsules, liquids, gels, syrups, slurries, suspensions and the like, for oral ingestion by a patient to be treated.

Pharmaceutical compositions suitable for use in the present invention include compositions wherein the antagonists of Rspo3 polypeptides or Rspo3 nucleic acids are contained in an effective amount to achieve its intended purpose. Determination of the effective amounts is well within the capability of those skilled in the art, especially in light of the detailed disclosure provided herein.

In addition to the antagonists of Rspo3 polypeptides or Rspo3 nucleic acids these pharmaceutical compositions may contain suitable pharmaceutically acceptable carriers comprising excipients and auxiliaries which facilitate processing of the antagonists of Rspondin into preparations which can be used pharmaceutically. The preparations formulated for oral administration may be in the form of tablets, dragees, capsules, or solutions.

The pharmaceutical compositions of the present invention may be manufactured in a manner that is itself known, by means of conventional mixing, dissolving, granulating, dragee-making, levigating, emulsifying, encapsulating, entrapping or lyophilizing processes.

Pharmaceutical formulations for parenteral administration include aqueous solutions of the antagonists of Rspo3 polypeptides or Rspo3 nucleic acids in water-soluble form. Additionally, suspensions of the agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the agents to allow for the preparation of highly concentrated solutions.

Pharmaceutical preparations for oral use can be obtained by combining the antagonists of Rspo3 polypeptides or Rspo3 nucleic acids with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of doses.

Pharmaceutical preparations which can be used orally include push-fit capsules made of gelatin, as well as soft, sealed capsules made of gelatin and a plasticizer, such as glycerol or sorbitol. The push-fit capsules can contain the active agents in admixture with filler such as lactose, binders such as starches, and/or lubricants such as talc or magnesium stearate and, optionally, stabilizers. In soft capsules, the active agants may be dissolved or suspended in suitable liquids, such as fatty oils, liquid paraffin, or liquid polyethylene glycols. In addition, stabilizers may be added.

Compositions comprising an antagonist of Rspo3 polypeptides or Rspo3 nucleic acids formulated in a compatible pharmaceutical carrier may be prepared, placed in an appropriate container, and labelled for treatment of osteoporosis and other conditions where treatment involves promoting bone formation and/or inhibiting bone resorption.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the active agents may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

For any antagonist of Rspo3 polypeptides or Rspo3 nucleic acids used in the method of the invention, the therapeutically effective dose can be estimated initially from cell culture assays. For example, a dose can be formulated in animal models to achieve a circulating concentration range that includes the IC₅₀ as determined in cell culture (i.e., the concentration of the test compound which achieves a half-maximal inhibition of the PIP activity). such information can be used to more accurately determine useful doses in humans.

A therapeutically effective dose refers to that amount of the antagonist of Rspo3 polypeptides or Rspo3 nucleic acids that results in amelioration of symptoms or a prolongation of survival in a patient. Toxicity and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Antagonists of Rspo3 polypeptides or Rspo3 nucleic acids which exhibit large therapeutic indices are preferred. The data obtained from these cell culture assays and animal studies can be used in formulating a range of dosage for use in human. The dosage of such antagonists of Rspo3 polypeptides or Rspo3 nucleic acids lies preferably within a range of circulating concentrations that include the ED50 with little or no toxicity. The dosage may vary within this range depending upon the dosage form employed and the route of administration utilized. The exact formulation, route of administration and dosage can be chosen by the individual physician in view of the patient's condition. (See e.g. Fingl et al., 1975, in “The Pharmacological Basis of Therapeutics”, Ch. 1 p 1).

Dosage amount and interval may be adjusted individually to provide plasma levels of the active agents which are sufficient to maintain the Rspo3 inhibitory effects. Usual patient dosages for systemic administration range from 1-2000 mg/day, commonly from 1-250 mg/day, and typically from 10-150 mg/day. Stated in terms of patient body weight, usual dosages range from 0.02-25 mg/kg/day, commonly from 0.02-3 mg/kg/day, typically from 0.2-1.5 mg/kg/day.

Dosage amount and interval may be adjusted individually to provide plasma levels of the active agents which are sufficient to maintain the Rspondin inhibitory or promoting effects. Usual average plasma levels should be maintained within 50-5000 μg/ml, commonly 50-1000 μg/ml, and typically 100-500 μg/ml.

Alternately, one may administer the active agents in a local rather than systemic manner, for example, via injection directly into a target site often in a depot or sustained release formulation.

Furthermore, one may administer the pharmaceutical composition in a targeted drug delivery system, for example, in a liposome coated with target-specific antibody. The liposomes will be targeted to and taken up selectively by the target site.

In cases of local administration or selective uptake, the effective local concentration of the pharmaceutical composition may not be related to plasma concentration.

The Rspo3 nucleic acids or compounds capable of binding to Rspo3 polypeptides or Rspo3 nucleic acids, such as antibodies or nucleotide probes, may be used for diagnostic purposes for detection of Rspo3 expression in low bone density disorders.

Reagents suitable for detecting Rspo3, such as Rspo3 nucleic acids or compounds capable of binding to Rspo3 polypeptides or Rspo3 nucleic acids may have a number of uses for the diagnosis of processes, conditions or diseases resulting from, associated with and/or accompanied by, aberrant expression of Rspo3. The diagnostic procedures are preferably carried out on samples obtained from a subject, e.g. a human patient, e.g. samples from body fluids such as whole blood, plasma, serum or urine, or tissue samples such as biopsy or autopsy samples. For example, the Rspo3 sequence may be used in amplification, e.g. hybridization assays to diagnose abnormalities of Rspondin expression; e.g., Southern or Northern analysis, including in situ hybridization assays.

Further, the present invention is explained in more detail by the following Examples.

### 6. EXAMPLES

### 6.1 Analysis Of Bone Phenotype In Rspo3+/− Animals

### 6.1.1 Materials And Methods

Rspo3^{+/-} animals and wild-type litter mate were generated as described by Kazanskaya et al. (Development. 2008, 135:3655-3664).

Bone phenotype was determined in 12-week-old Rspo3^{+/-} and wildtype littermate mice. Mouse tibias were recovered from 12-week-old mice following sacrifice and were used for tomodensitometry. For tomodensitometry, right tibias were fixed overnight in 3.7% formaldehyde in PBS, washed in PBS, and then stored in 70% ethanol. Micro-CT (μCT) scans the metaphyseal region were performed at an isotropic resolution of 9 μm, to obtain trabecular bone structural parameters. Using a two- and three-dimensional model and a semiautomatic contouring algorithm, we determined three-dimensional bone volume, bone surface, and the trabecular thickness. Three-dimensional images were obtained on a Scanco Medical micro-CT scanner CT (μCT 20; Scanco Medical AG, Bassersdorf, Switzerland). A total of 450 images were obtained from each bone sample using a 512×512 matrix, resulting in an isotropic voxel resolution of 18×18×18 μm³. Measurements were stored in three-dimensional (3D) image arrays with an isotropic voxel size of 9 μm. A constrained 3D Gaussian filter was used to partly suppress the noise in the volumes. The bone tissue was segmented from marrow using a global thresholding procedure.

### 6.1.2 Results

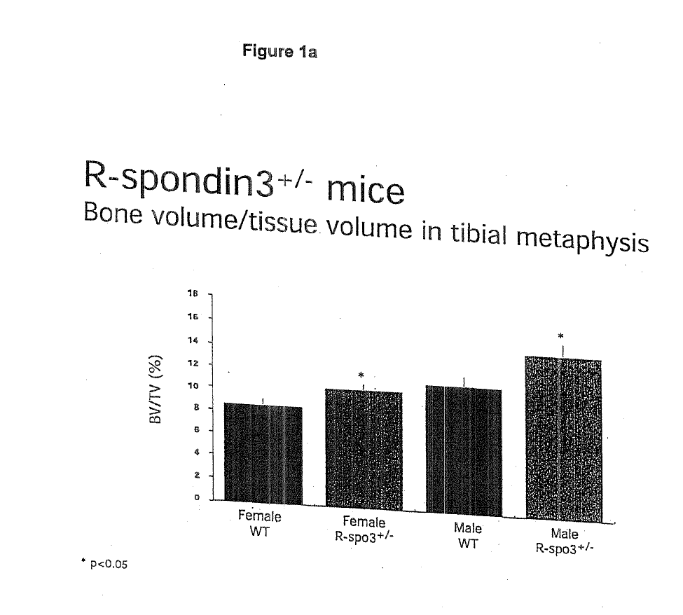

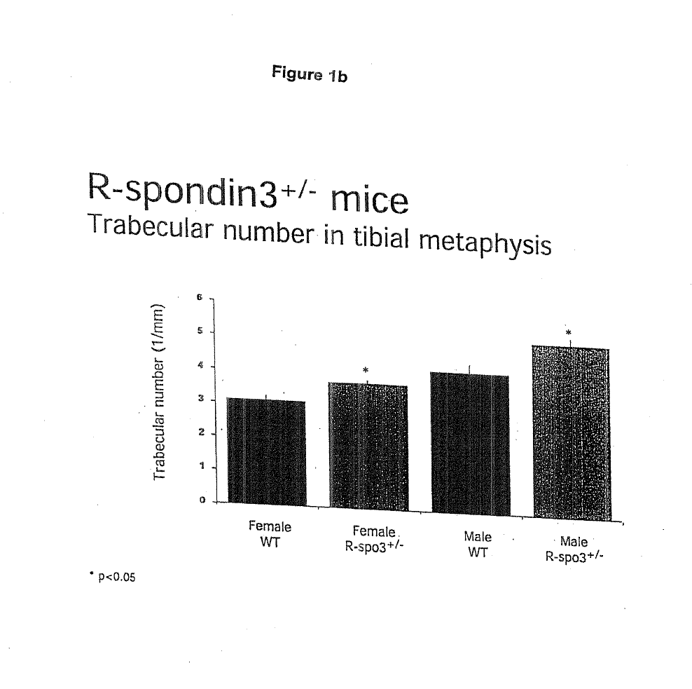

Tibias from 12-week-old animals were analysed by μCT for bone volume. Analysis of bones from single Rspo3^{+/-} mice showed a significant increase in bone volume compared to wild type littermates and in both genders (see FIG. 1a). In addition to the increase in bone volume we could demonstrate an increase in trabecular numbers in mutant mice compared to wild type littermates (see FIG. 1b). Collectively these data indicate that deleting one copy of Rspo3 and thus impairing its function result in increased bone mass.

### 6.2 Analysis of Bone Formation and Bone Resorption Markers in Rspo3+/− Animals

### 6.2.1 Materials and Methods

Rspo3^{+/-} animals and wild-type litter mates were generated as described by Kazan-skaya et al. (Development. 2008, 135:3655-3664). For osteocalcin and TRACP 5b (also known as TRAP) level measurements, blood was collected from in 12-week-old Rspo3^{+/-} and wildtype littermate mice. TRACP 5b measurements were performed using the MouseTRAP™ kit supplied by ImmunoDiagnostic Systems Inc, following the protocol supplied (SB-TR103). Briefly, the MouseTRAP™ Assay uses a poly-clonal antibody prepared using recombinant mouse TRACP as antigen. In the test, the antibody is incubated in anti-rabbit IgG-coated microtiter wells. After washing, standard, control, and samples are incubated in the walls, and hound TRACP 5b activity is determined with a chromogenic substrate to develop colour. The reaction is stopped, and the absorbance of the reaction mixture is read in a microtiter plate reader, colour intensity being directly proportional to the amount and activity of TRACP 5b present in the sample. Serum osteocalcin was assayed with kits and re-agents from Biomedical Technologies Inc. (Stoughton, Mass., USA) as previously described (Sims, N. A., Clement-Lacroix, P., Minet, D., Fraslon-Vanhulle, C., Gaillard-Kelly, M., Resche-Rigon, M. & Baron, R. (2003) J Clin Invest 111, 1319-27).

### 6.2.2 Results

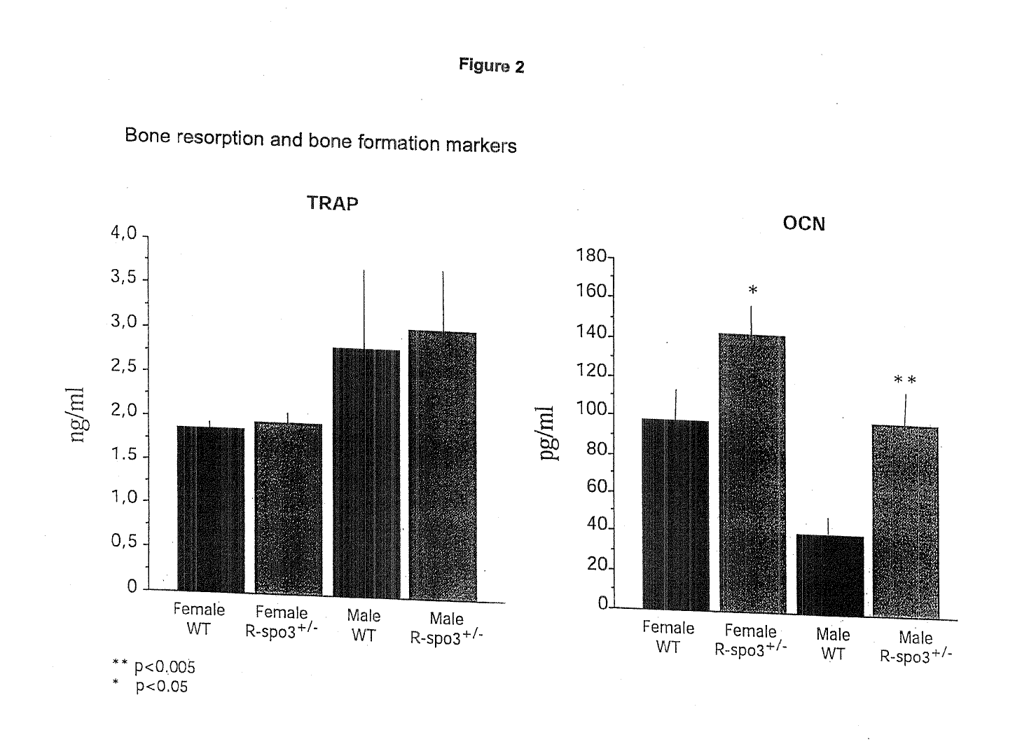

Osteocalcin and TRACP 5b are well known serum markers for bone formation and bone resorption, respectively. Our data show that TRACP 5b level in comparable in Rspo3^{+/-} and wild-type litter mate animals. In contrast, osteocalcin level in significantly higher in Rspo3^{+/-} animals compared to wild-type litter mates. This increases in osteoclacin in Rspo3^{+/-} animals was confirmed in both genders. These data clearly demonstrate that deletion of on copy of Rspo3 and thus impairing Rspo3 function results in increased bone formation with no detectable effect on bone resorption (see FIG. 2).

The present invention is not to be limited in scope by the exemplified embodiments which are intended as illustrations of single aspects of the invention, and any clones, DNA or functionally equivalents to Rspondin are within the scope of the invention. Indeed, various modifications of the invention in addition to those described herein will become apparent to those skilled in the art from the foregoing description and accompanying drawings. Such modifications are intended to fall within the scope of the appended claims.

All references cited herein are hereby incorporated by reference in their entirety.

### 6.3 Measuring Rspo3 Activation of the Wnt/PCP Pathway

### 6.3.1 Materials and Methods

Xenopus embryos were microinjected in 4 blastomers at the 4-cell stage with a Jun-responsive ATF luciferase reporter (100 pg), Renilla luciferase plasmid pRL (75 pg), and coinjected with mRNA encoding Fz7 (250 pg), Wnt5A (250 pg or 500pg), or Rspo3 (400 pg or 800 pg). 10 embryos each were collected at early neurula st.13 and homogenized in 150 μl of passive lysis buffer (Promega). Firefly luciferase and Renilla luciferase activity were determined in a fluorometer. The ATF reporter activities were normalized to Renilla activities and the basal value at st.13 in embryos was set as 1.0.

### 6.3.2 Results

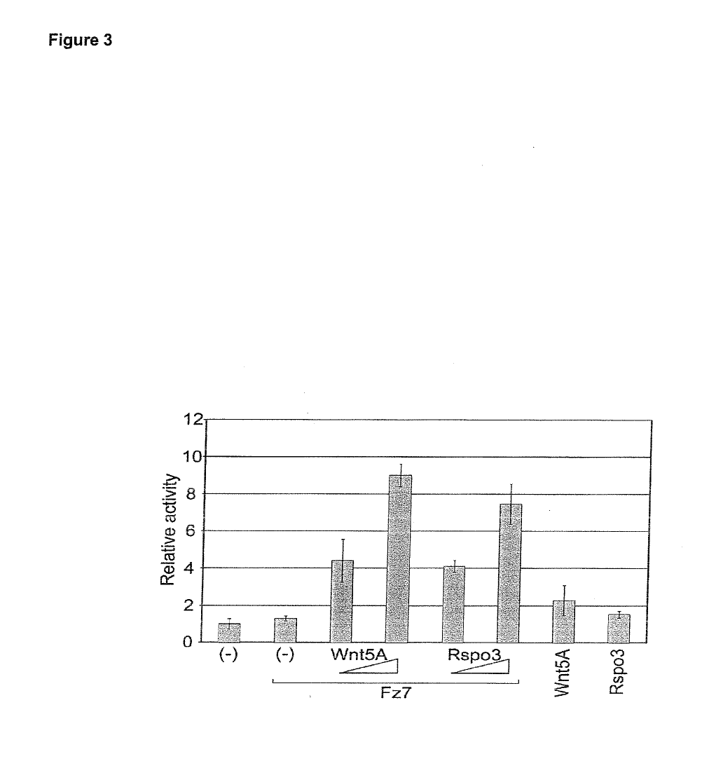

Wnt5a and Fz7 are well known activators of the Wnt/PCP pathway. They xynergistically activate a Jun responsive luciferase reporter in Xenopus embryos (FIG. 3). Likewise, Rspo3 is able to activate the reporter with Fz7. This assay can be used to screen for Rspo3 modulators and inhibitors.

1. Use of an antagonist of a Rspondin-3 (Rspo3) polypeptide or a Rspondin-3 (Rspo3) nucleic acid for the manufacture of a medicament for the promotion of bone formation and/or inhibition of bone resorption.

2. The use of claim 1 for the prevention or treatment of a low bone density disorder.

3. The use of claim 1 for the prevention or treatment of osteoporosis, bone metastasis or multiple myeloma.

4. The use of claim 1 wherein the Rspo3 antagonist is an anti-Rspo3 antibody.

5. The use of claim 1 wherein the Rspo3 antagonist is a nucleic acid molecule capable of inhibiting Rspo3 translation, transcription, expression and/or activity.

6. The use of claim 5 wherein the Rspo3 antagonist is an antisense molecule, shRNA molecule or siRNA molecule.

7. A method for promoting bone formation or inhibiting bone resorption comprising administering to a subject in need thereof a therapeutically effective dose of a Rspo3 antagonist.

8. The method of claim 7, wherein the subject is human.

9. A method for the diagnosis or monitoring of bone-associated processes, conditions or disorders, comprising determining the amount, activity and/or expression of an Rspo3 polypeptide or Rspo3 nucleic acid in a sample.

10. The method according to claim 9, wherein the method comprises the additional step of comparing the amount, activity and/or expression of said Rspo3 polypeptide or Rspo3 nucleic acid to the amount, activity and/or expression of said Rspo3 polypeptide or Rspo3 nucleic acid in a control sample.

11. The method according to any one of claims 9 to 10 claim 9 wherein the sample is a body fluid or tissue sample.

12. The method according to claim 9, wherein an increased amount, activity and/or expression of said Rspo3 polypeptide or Rspo3 nucleic acid is associated with increased bone resorption and/or decreased bone formation.

13. Use of an Rspo3 polypeptide, an Rspo3 nucleic acid to evaluate and/or screen test compounds for their ability to modulate bone-associated processes, conditions or disorders, wherein an increased activity of said Rspo3 polypeptide or Rspo3 nucleic acid in the presence of the test compound when compared to a control is associated with increased bone resorption and/or decreased bone formation.

14. Use of an Rspo3 polypeptide or an Rspo3 nucleic acid to evaluate and/or screen test compounds for their ability to modulate bone-associated processes, conditions or disorders, wherein a decreased activity of said Rspo3 polypeptide or Rspo3 nucleic acid in the presence of the test compound when compared to a control is associated with decreased bone resorption and/or increased bone formation.

15. Use of a recombinant cell which expresses Rspo3 or non-human transgenic organism exhibiting modified Rspo3 expression to evaluate and/or screen test compounds for their ability to modulate bone-associated processes, conditions or disorders, wherein an increased amount, activity and/or expression of said Rspo3 polypeptide or Rspo3 nucleic acid is associated with increased bone resorption and/or decreased bone formation.

16. Use of a recombinant cell which expresses Rspo3 or non-human transgenic organism exhibiting modified Rspo3 expression to evaluate and/or screen test compounds for their ability to modulate bone-associated processes, conditions or disorders, wherein a decreased amount, activity and/or expression of said Rspo3 polypeptide or Rspo3 nucleic acid is associated with decreased bone resorption and/or increased bone formation.


SEQUENCE LISTING (2 sequences)

SEQ ID NO: 1 - protein, 272 aa, Homo sapiens
  PEPTIDE (1)..(272): human Rspondin-3
MHLRLISWLFIILNFMEYIGSQNASRGRRQRRMHPNVSQGCQGGCATCSDYNGCLSCKPRLFFALERIGMKQIGVCLSSCPSGYYGTRYPDINKCTKCKADCDTCFNKNFCTKCKSGFYLHLGKCLDNCPEGLEANNHTMECVSIVHCEVSEWNPWSPCTKKGKTCGFKRGTETRVREIIQHPSAKGNLCPPTNETRKCTVQRKKCQKGERGKKGRERKRKKPNKGESKEAIPDSKSLESSKEIPEQRENKQQQKKRKVQDKQKSVSVSTVH

SEQ ID NO: 2 - DNA, 2165 bp, Homo sapiens
  gene (1)..(2165): human Rspondin-3
gcggccgccccggcggctcctggaaccccggttcgcggcgatgccagccaccccagcgaagccgccgcagttcagtgcttggataatttgaaagtacaatagttggtttccctgtccacccgccccacttcgcttgccatcacagcacgcctatcggatgtgagaggagaagtcccgctgctcgggcactgtctatatacgcctaacacctacatatattttaaaaacattaaatataattaacaatcaaaagaaagaggagaaaggaagggaagcattactgggttactatgcacttgcgactgatttcttggctttttatcattttgaactttatggaatacatcggcagccaaaacgcctcccggggaaggcgccagcgaagaatgcatcctaacgttagtcaaggctgccaaggaggctgtgcaacatgctcagattacaatggatgtttgtcatgtaagcccagactattttttgctctggaaagaattggcatgaagcagattggagtatgtctctcttcatgtccaagtggatattatggaactcgatatccagatataaataagtgtacaaaatgcaaagctgactgtgatacctgtttcaacaaaaatttctgcacaaaatgtaaaagtggattttacttacaccttggaaagtgccttgacaattgcccagaagggttggaagccaacaaccatactatggagtgtgtcagtattgtgcactgtgaggtcagtgaatggaatccttggagtccatgcacgaagaagggaaaaacatgtggcttcaaaagagggactgaaacacgggtccgagaaataatacagcatccttcagcaaagggtaacctgtgtcccccaacaaatgagacaagaaagtgtacagtgcaaaggaagaagtgtcagaagggagaacgaggaaaaaaaggaagggagaggaaaagaaaaaaacctaataaaggagaaagtaaagaagcaatacctgacagcaaaagtctggaatccagcaaagaaatcccagagcaacgagaaaacaaacagcagcagaagaagcgaaaagtccaagataaacagaaatcggtatcagtcagcactgtacactagagggttccatgagattattgtagactcatgatgctgctatctcaaccagatgcccaggacaggtgctctagccattaggaccacaaatggacatgtcagttattgctctgtctaaacaacattcccagtagttgctatattcttcatacaagcatagttaacaacaaagagccaaaagatcaaagaagggatactttcagatggttgtcttgtgtgcttctctgcatttttaaaagacaagacattcttgtacatattatcaataggctataagatgtaacaacgaaatgatgacatctggagaagaaacatcttttccttataaaaatgtgttttcaagctgttgttttaagaagcaaaagatagttctgcaaattcaaagatacagtatcccttcaaaacaaataggagttcagggaagagaaacatccttcaaaggacagtgttgttttgaccgggagatctagagagtgctcagaattagggcctggcatttggaatcacaggatttatcatcacagaaacaactgttttaagattagttccatcactctcatcctgtatttttataagaaacacaagagtgcataccagaattgaatataccatatgggattggagaaagacaaatgtggaagaaatcatagagctggagactacttttgtgctttacaaaactgtgaaggattgtggtcacctggaacaggtctccaatctatgttagcactatgtggctcagcctctgttaccccttggattatatatcaacctgtaaacatgtgcctgtaacttacttccaaaaacaaaatcatacttattagaagaaaattctgattttatagaaaaaaaatagagcaaggagaatataacatgtttgcaaagtcatgtgttttctttctcaatgagggaaaaacaattttattacctgcttaatggtccacctggaactaaaagggatactattttctaacaaggtatatctagtaggggagaaagccaccacaataaatatatttgttaatagtttttcaaaaaaaaaaaaaaaaaaaaaaaaaa